(19) United States
(12) Patent Application Publication
(10) Pub. No.: US 2026/0265698 A1
(43) Pub. Date: Sep. 10, 2026

(54) SYSTEM AND METHOD FOR CAPILLARY BLOOD STEM CELL REPROGRAMMING USING UPPER-ARM BLOOD COLLECTION DEVICES FOR PATIENT-SPECIFIC DISEASE MODELING

(71) Applicant: Mohammed Harris, Basking Ridge, NJ (US)

(72) Inventor: Mohammed Harris, Basking Ridge, NJ (US)

(73) Assignee: TimepointDx, Basking Ridge, NJ (US)

(21) Appl. No.: 19/070,450

(22) Filed: Mar. 4, 2025

(51) Int. Cl. C12N 5/074; A61B 5/15; C12N 5/00; G01N 33/50

(52) U.S. Cl. CPC C12N 5/0696; A61B 5/150022; C12N 5/0018; G01N 33/502; G01N 33/5073; C12N 2501/602; C12N 2501/603; C12N 2501/604; C12N 2501/606; C12N 2501/608; C12N 2503/02; C12N 2506/11; G01N 2500/10; G01N 2800/52

(57) ABSTRACT

The present invention discloses a system and method for generating patient-specific induced pluripotent stem cells (iPSCs) from capillary blood collected via upper-arm blood collection devices. The process utilizes specialized devices such as Tasso, Yourbio Health TAP, RedDrop Dx, and Impress to obtain capillary blood without venipuncture, isolates peripheral blood mononuclear cells (PBMCs), and reprograms them into iPSCs using defined factors. The resulting patient-specific iPSCs retain the molecular characteristics of the original donor, enabling advanced in vitro disease modeling, drug discovery, and therapeutic applications. The system integrates artificial intelligence for predictive analysis and supports multiomics studies. This innovation enables “clinical trials in a dish” with patient-matched cells, providing powerful tools for personalized medicine approaches without the ethical concerns associated with embryonic stem cells.

### FIELD OF THE DISCLOSURE

The present invention relates to regenerative medicine, specifically to systems and methods for generating patient-specific induced pluripotent stem cells (iPSCs). More particularly, the invention pertains to an integrated approach for deriving iPSCs from capillary blood collected via upper-arm blood collection devices, and their subsequent application in personalized disease modeling, drug discovery, and therapeutic development.

### BACKGROUND OF THE INVENTION

### History of Regenerative Medicine

The concept of regenerative medicine, while seemingly novel, has deep historical and cultural roots. Examples of regeneration can be found in mythology, such as Prometheus whose liver would regrow after being pecked by eagles, as well as in nature, where certain animals like lizards and crocodiles demonstrate exceptional regenerative abilities. These observations have fascinated scientists for generations, suggesting the inherent potential for tissue regeneration in biological systems (Zaret et al., 2011).

The scientific pursuit of regenerative medicine gained momentum in the 20th century, driven by significant healthcare challenges including the rise in degenerative diseases due to lifestyle changes, growing demands from an aging population, and limited availability of donor organs. Traditional medical interventions were largely palliative, targeting symptoms rather than restoring functional tissue (Takahashi & Yamanaka, 2006).

A pivotal moment in the field occurred in 1961 when scientists Till and McCulloch discovered the self-renewal capacity of living cells. During experiments with irradiated mice injected with fresh donor bone marrow cells, they observed clonal growth of the injected cells, which proved key to the mice's survival. These findings led to the definition of stem cells as mother cells capable of self-division and differentiation into multiple cell types (Till & McCulloch, 1961).

The Rise of Induced Pluripotent Stem Cells (iPSCs)

Prior to 2006, stem cell research faced significant challenges. Human embryonic stem cells, though possessing the highest differentiation potential, raised substantial ethical concerns due to the requirement of sacrificing early embryos. Meanwhile, adult stem cells, though ethically less problematic, were limited by their restricted differentiation potential and the difficulties in isolating and expanding them to sufficient quantities for therapeutic applications (Takahashi & Yamanaka, 2007).

The landmark achievement came in 2006 when Takahashi and Yamanaka introduced induced pluripotent stem cells (iPSCs). These cells were generated through reprogramming somatic cells and demonstrated exceptional self-differentiating abilities similar to embryonic stem cells. This remarkable aptitude was confirmed by the presence of factors like OCT-4, SOX-2, KLF-4, and c-Myc (Takahashi & Yamanaka, 2006). iPSCs offered several advantages over traditional stem cell types. They eliminated ethical concerns associated with embryonic stem cells while maintaining comparable differentiation potential. Additionally, they reduced the risk of immune rejection during transplantation, as they could be derived from a patient's own cells (Guha et al., 2013). Their vast differentiation potential made them ideal for toxicity studies, high-throughput drug screening, and modeling therapeutic responses through in vitro systems (Wobus & Loser, 2011).

### Early Methods and Limitations

The initial breakthrough in iPSC generation involved reprogramming adult mouse skin fibroblasts using recombinant retroviruses to transduce four major transcription factors: OCT4, SOX2, KLF4, and c-Myc. The reprogrammed cells expressed pluripotency markers OCT-4 and Nanog and could contribute to entire germline formation in chimeric mice. This technique was soon extended to human dermal fibroblasts (Takahashi et al., 2007).

These early methods, while revolutionary, presented significant limitations. The procurement of skin biopsies required invasive procedures, causing discomfort and scarring, particularly problematic for elderly or pediatric populations (Maherali et al., 2007). The reprogramming efficiency of human dermal fibroblasts was extremely low, typically 0.01% to 0.1%, due to strong epigenetic barriers (Takahashi et al., 2007). Additionally, dermal fibroblasts proliferate slowly, risking cellular senescence and limiting the availability of cultured cells. Concerns were also raised about potential mutations due to long-term UV exposure of dermal cells, potentially limiting the clinical efficacy of the generated iPSCs (Van der Pols et al., 2006).

The use of retroviral and lentiviral vectors for reprogramming introduced further complications, including the risk of insertional mutagenesis and potential tumor formation through reactivation of exogenous transgenes (Okita et al., 2007). These limitations highlighted the need for alternative approaches that could improve safety, feasibility, and efficiency of iPSC generation for therapeutic applications (Yu et al., 2007).

### The Promise of Blood-Derived iPSCS

In the search for more accessible and less invasive sources for iPSC generation, peripheral blood emerged as a promising alternative to skin fibroblasts. Blood collection is standardized, less invasive, and less traumatic compared to skin biopsies (Zhang et al., 2013). Moreover, blood samples provide larger quantities of cells at early passages, ensuring less batch variability and genomic variation compared to the limited yield from a 4 mm skin punch biopsy (S. Rab et al., 2014).

Despite these advantages, traditional blood collection and processing methods present their own challenges. The heterogeneous nature of blood samples results in mixed populations containing monocytes, lymphocytes (T cells, B cells, NK cells), dendritic cells, platelets, and granulocytes, which are difficult to separate using standard density gradient isolation methods (Boyum, 1968; Slitchter, 1981; Schakel et al., 1996). Factors such as sample handling, transportation, centrifugation conditions, and donor variability can compromise cell quality and yield (Ferrant & Thong, 1980).

### Advances in Cell Isolation and Reprogramming

To address these challenges, researchers have developed automated cell separation systems such as AutoMACS® Pro Separator (Miltenyi Biotec) and Sepax™ (Biosafe) for more efficient isolation of peripheral blood mononuclear cells (PBMCs). Magnetic-activated cell sorting (MACS) has emerged as a widely accepted approach to refine PBMC yield through selective depletion of undesired cells and enrichment of targeted populations (Miltenyi et al., 2017).

Microfluidic-based isolation techniques have also been developed to separate monocytes, lymphocytes, and other PBMNC populations from granulocytes and erythrocytes with higher efficiency (Chen et al., 2019). Additionally, optimized culture conditions using serum-free formulations with selective growth factors like IL-2 and IL-4, and extracellular matrices such as collagen or fibronectin, have been proposed for enhanced cellular proliferation and viability (Zhou et al., 2018; Zhou et al., 2020).

Parallel to these advancements in cell isolation, non-integrative reprogramming methods have been developed to overcome the risks associated with traditional viral vector approaches. Sendai virus (SeV), an enveloped, non-pathogenic, single-stranded RNA virus that operates at the cytoplasmic level, avoids genomic integration and has become a promising alternative for generating integration-free iPSCs (Schlaeger et al., 2015).

Other non-integrative approaches include episomal vectors, which can deliver reprogramming factors transiently while being diluted or degraded over successful cellular divisions (Kumar et al., 2018; Sridhar et al., 2016). RNA-based delivery systems, where synthetic mRNA encoding reprogramming factors is used for direct reprogramming, offer perhaps the safest method due to the shorter life of RNA molecules, though they require multiple transfections (Omole and Fakoya, 2018).

Chemical-based reprogramming techniques have also gained attention, employing compounds like valproic acid (a histone deacetylase inhibitor), 5′ azacytidine (a DNA methyltransferase inhibitor), and GSK3β inhibitors like CHIR99021 that promote chromatin remodeling, making somatic cells more receptive to pluripotency induction (Choi et al., 2015).

### The Challenge of Capillary Blood and Device-Specific Collection

Despite these advances, a significant gap remains in the field: the effective utilization of capillary blood for iPSC generation. Traditional iPSC protocols rely primarily on venous blood, which requires phlebotomist intervention and often yields excessive sample volumes for personalized applications. Capillary blood, which can be collected through less invasive means, has been underexplored due to challenges in collection methods, limited volume, and processing complications.

The emergence of specialized upper-arm blood collection devices presents a unique opportunity to address this gap. Devices such as Tasso, YourBio Health TAP Micro, RedDropDx, and Impress enable the collection of capillary blood samples without the need for phlebotomist intervention. However, no standardized protocol exists for generating iPSCs from capillary blood collected through these devices.

Additionally, the current landscape lacks integrated systems that seamlessly connect capillary blood collection from upper-arm devices to PBMC isolation and subsequent iPSC generation while maintaining patient-specific characteristics throughout the process. This gap is particularly significant for applications requiring personalized, patient-specific in vitro models for disease modeling, drug discovery, and therapeutic development.

### Applications and Future Potential

The potential applications of blood-derived iPSCs are vast. In cell and gene therapies, these patient-specific iPSCs can potentially treat blood disorders like leukemia and aplastic anemia with higher efficiency and more feasible outcomes (Gustafsson et al., 2015; Dhaenens et al., 2019). As disease models, they enable researchers to study specific disease mechanisms and develop personalized treatment strategies (Chung et al., 2013).

Integration with organ-on-a-chip models represents another frontier, where iPSCs help replicate physiological conditions of organs for more accurate drug screening and toxicity analysis, potentially reducing animal testing in preclinical settings (Bhatia & Ingber, 2014).

### Object of the Invention

In light of these challenges and opportunities, there is a clear need for a novel system and method specifically designed for generating patient-specific iPSCs from capillary blood collected via upper-arm collection devices. Such a system would bridge the significant gap between the potential of less invasive capillary blood collection and the growing demand for patient-specific iPSCs in personalized medicine.

The object of the present invention is therefore to provide an integrated system that enables the collection of capillary blood from upper-arm devices, efficient isolation of PBMCs from these limited samples, and effective reprogramming into patient-specific iPSCs that retain the donor's molecular characteristics. This system aims to facilitate advanced in vitro disease modeling, drug discovery, and therapeutic applications without the need for venipuncture or phlebotomist intervention, thereby democratizing access to personalized stem cell technologies.

### SUMMARY OF THE INVENTION

The present invention relates to a novel system and method for generating patient-specific induced pluripotent stem cells (iPSCs) from capillary blood collected using upper-arm blood collection devices, and utilizing said iPSCs for advanced in vitro modeling, drug discovery, and therapeutic applications. The invention is specifically directed to the unique application of capillary blood, as opposed to venous blood, for the isolation of peripheral blood mononuclear cells (PBMCs), which are subsequently reprogrammed into iPSCs. The generated iPSCs retain the molecular characteristics of the patient, enabling highly accurate and personalized in vitro modeling for a wide range of applications, including disease modeling, drug screening, toxicology analysis, and predictive clinical trial simulations.

The system comprises an upper-arm blood collection device configured to collect capillary blood from a patient. The upper-arm blood collection device is selected from the group consisting of Tasso, Yourbio Health TAP Micro, RedDropDx, and Impress. These devices are specifically designed to collect capillary blood without the need for venipuncture or phlebotomist intervention, thereby enabling a minimally invasive and patient-friendly blood collection process. The collected capillary blood is then processed to isolate PBMCs using a processing module, which may include a collection tube coupled to paramagnetic beads for red blood cell separation and pulldown, a centrifugation unit for mechanical separation, or a cell separation reagent for chemical separation. The isolated PBMCs are subsequently reprogrammed into iPSCs using a reprogramming module, which may include a mechanoporation system or a non-transmissible or transmissible vector system, which introduces a defined set of reprogramming factors, such as OCT4, SOX2, KLF4, MYC, and LIN28 as well as custom factors on the basis of Yamanaka factors, most predominantly packaged as circularized or linearized delivery cargo.

The generated iPSCs are utilized in an in vitro modeling module, which is configured to conduct patient-specific disease modeling, drug discovery, and therapeutic applications. The in vitro modeling module may integrate advanced technologies, such as artificial intelligence (AI), for predictive analysis of drug efficacy, pharmacokinetics, and pharmacodynamics. Additionally, the module may perform multiomics analysis, including genomics, transcriptomics, proteomics, and metabolomics, to provide a comprehensive understanding of the patient-specific molecular characteristics. The module may further incorporate a model for conducting “clinical trial in a dish”, hence live in vitro cell culture with passaging and maintenance as desired. Such modules can run prior to clinical trial studies (i.e. prior to investigational drug applications, or IND, primarily for producing in vitro data) or in parallel with clinical trial studies (i.e. post-IND and during Phase 1, 2, and 3 of clinical trials).

The method of the invention comprises the steps of collecting capillary blood from a patient using an upper-arm blood collection device, isolating PBMCs from the collected capillary blood, reprogramming the isolated PBMCs into iPSCs using a defined set of reprogramming factors, and utilizing the generated iPSCs for patient-specific in vitro modeling, drug discovery, or therapeutic applications. The method is characterized by its use of capillary blood, which is collected in a minimally invasive manner, and its ability to generate iPSCs that retain the molecular characteristics of the patient. This patient-specific approach enables highly accurate and personalized in vitro modeling, which can be used to predict safety and efficacy endpoints for clinical trials, conduct drug screening, and develop personalized therapeutics.

The invention is particularly advantageous in that it leverages the unique properties of capillary blood, which is more accessible and less invasive to collect than venous blood. The use of upper-arm blood collection devices further enhances the convenience and patient compliance of the blood collection process. The generated iPSCs are highly representative of the patient's molecular characteristics, making them ideal for a wide range of applications, including advanced disease modeling, drug discovery, and translational research. The integration of AI and multiomics analysis further enhances the predictive power and utility of the in vitro modeling module, enabling more accurate and comprehensive analysis of drug efficacy, pharmacokinetics, and pharmacodynamics.

As such, the present invention provides a novel and highly effective system and method for generating patient-specific iPSCs from capillary blood collected using upper-arm blood collection devices. The invention is characterized by its minimally invasive blood collection process, its ability to generate highly representative iPSCs, and its integration of advanced technologies for predictive analysis and personalized applications. The invention has broad applications in the fields of disease modeling, drug discovery, and therapeutic development, and represents a significant advancement in the field of regenerative medicine and personalized therapeutics.

### BRIEF DESCRIPTION OF THE DRAWINGS

The accompanying drawings, which are incorporated in and constitute a part of this specification, illustrate embodiments of the invention and, together with the description, serve to explain the principles of the invention. In the drawings:

FIG. 1 is a schematic illustration showing an upper-arm blood collection device positioned on a user according to one embodiment of the present invention.

FIG. 2 is a flow diagram illustrating the complete Capillary Stem Cell Reprogramming (CSCR) process from blood collection through downstream applications, including reprogramming, differentiation, disease modeling, drug screening, and discovery of therapeutic agents.

### DETAILED DESCRIPTION OF PREFERRED EMBODIMENTS

The following description of preferred embodiments refers to the accompanying drawings, which illustrate specific embodiments of the invention. Other embodiments having different structures and operations do not depart from the scope of the present invention. The same reference numbers may be used in the drawings and the following description to refer to the same or like parts.

As used herein, the terms “comprising,” “including,” “containing,” “characterized by,” and grammatical equivalents thereof are inclusive or open-ended and do not exclude additional, unrecited elements or method steps, unless otherwise stated. Other than in the operating examples, or where otherwise indicated, all numbers expressing quantities, processing parameters, assessment scores, and so forth used in the specification and claims are to be understood as being modified in all instances by the term “about,” meaning within a reasonable range of the indicated value. The terms “a” and “an” refer to one or more of the elements described, whereas the term “plurality” refers to two or more of the elements described, unless the context clearly indicates otherwise.

The Capillary Stem Cell Reprogramming (CSCR) system and method described herein provide novel solutions for generating patient-specific induced pluripotent stem cells (iPSCs) from capillary blood collected using minimally invasive upper-arm blood collection devices. The invention incorporates advanced blood processing techniques, reprogramming protocols, and in vitro modeling methodologies to enable highly accurate and personalized applications in disease modeling, drug discovery, and therapeutic development. The following detailed description, along with the accompanying drawings, provides a comprehensive understanding of the various embodiments and aspects of the invention.

This invention addresses the challenges of obtaining patient-specific stem cells for in vitro modeling in a minimally invasive and patient-friendly manner. By leveraging capillary blood collected from upper-arm devices such as Tasso, Yourbio Health TAP Micro, RedDropDx, and Impress, the invention eliminates the need for venipuncture and phlebotomist intervention, thereby improving patient compliance and accessibility. The system and method further integrate advanced technologies, including artificial intelligence (AI) and multiomics analysis, to enhance the predictive power and utility of the generated iPSCs for personalized applications.

The present invention provides a comprehensive system and method for generating patient-specific iPSCs from capillary blood, comprising: an upper-arm blood collection device configured to collect capillary blood in a minimally invasive manner; a processing module for isolating peripheral blood mononuclear cells (PBMCs) from the collected capillary blood; a reprogramming module for converting the isolated PBMCs into iPSCs using defined reprogramming factors; and an in vitro modeling module for utilizing the generated iPSCs in patient-specific disease modeling, drug discovery, and therapeutic applications.

The invention's ability to generate iPSCs that retain the molecular characteristics of the patient represents a significant advancement in the field of regenerative medicine and personalized therapeutics. By enabling highly accurate in vitro modeling, the invention supports a wide range of applications, including advanced disease modeling, drug screening, toxicology analysis, pharmacokinetics and pharmacodynamics, target discovery, and personalized therapeutics modeling. The integration of AI and multiomics analysis further enhances the predictive capabilities of the system, enabling more accurate and comprehensive analysis of drug efficacy and safety.

The following detailed description, along with the accompanying drawings, provides a comprehensive understanding of the various embodiments and aspects of the invention, including the structure and operation of the system, the steps of the method, and the applications of the generated iPSCs. Specific embodiments are described to illustrate the invention, but it is understood that other embodiments with different structures and operations do not depart from the scope of the present invention.

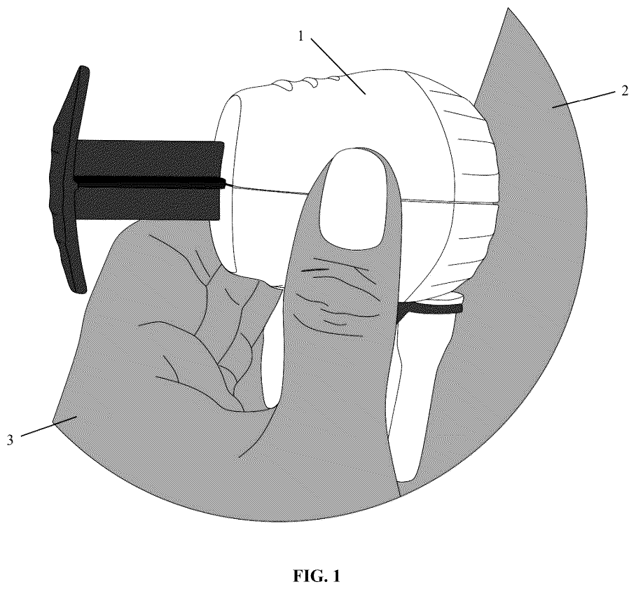

Referring now to FIG. 1, a schematic representation of the blood collection system according to one embodiment of the present invention is illustrated. FIG. 1 depicts an upper-arm blood collection device (1) positioned on the upper arm (2) of a person or user (3). The upper-arm blood collection device (1) may be selected from commercially available devices including, but not limited to, Tasso, YourBio Health TAP Micro, RedDropDx, and Impress devices, though other similar devices may be employed without departing from the scope of the present invention.

The upper-arm blood collection device (1) may comprise a housing with an adhesive surface configured to temporarily adhere to the skin of the upper arm (2). The device may further include a microfluidic channel system or an open gravity-directed system for directing the flow of collected capillary blood, a collection reservoir for temporary storage of the collected blood sample, and optionally, a vacuum or negative pressure mechanism to facilitate blood extraction through the skin without traditional venipuncture. The device (1) may be designed with a form factor optimized for upper arm (2) placement, considering the anatomical contours and capillary blood accessibility in this region.

In operation, the upper-arm blood collection device (1) may be applied to a prepared area of the upper arm (2) by the person (3) themselves, without requiring specialized training or the intervention of a phlebotomist. The device (1) may utilize microneedles, microfluidic channels, lancet-like blades or other minimally invasive mechanisms to access capillary blood vessels beneath the epidermis of the upper arm (2). These mechanisms may be specifically designed to target capillary vessels rather than venous structures, thereby ensuring the collection of true capillary blood which has been identified as particularly suitable for the subsequent PBMC isolation and iPSC generation processes described herein.

The person (3) may be seated or in another comfortable position during the blood collection process. The upper-arm blood collection device (1) may be configured to collect a predetermined volume of capillary blood, typically ranging from approximately 100 μL to 1 mL, though the exact volume may vary depending on the specific application requirements. The collection process may be completed within approximately 2 to 10 minutes, representing a significant time efficiency compared to traditional venipuncture methods.

The device (1) may incorporate visual or tactile indicators to inform the person (3) when sufficient blood volume has been collected. Upon completion of collection, the device (1) may automatically seal the collected sample to maintain sample integrity and prevent contamination. The device (1) may be equipped with preservation agents or stabilizers pre-loaded within the collection reservoir to maintain PBMC viability during temporary storage or transport.

The upper-arm blood collection device (1) may additionally incorporate temperature regulation elements to maintain optimal conditions for preserving cellular components during and immediately following collection. These elements may include passive thermal insulators or active temperature control mechanisms, depending on the specific embodiment of the invention.

The device (1) may be configured with secure sample transfer mechanisms that enable direct connection to the processing module of the system, thereby minimizing sample handling and potential contamination risks. This integration may be achieved through standardized connection ports or sealed transfer systems that maintain the closed nature of the collection and processing workflow.

The upper-arm blood collection device (1) may further include identification and tracking features, such as RFID tags, barcodes, or other identifiers that enable the system to maintain chain of custody throughout the entire workflow from blood collection through iPSC generation and subsequent in vitro modeling applications. These tracking features may help ensure that the patient-specific nature of the samples is maintained throughout all processing steps.

Through this configuration, the upper-arm blood collection device (1) enables the minimally invasive collection of capillary blood directly from the person (3), establishing the critical first step in the patient-specific iPSC generation workflow that characterizes the present invention.

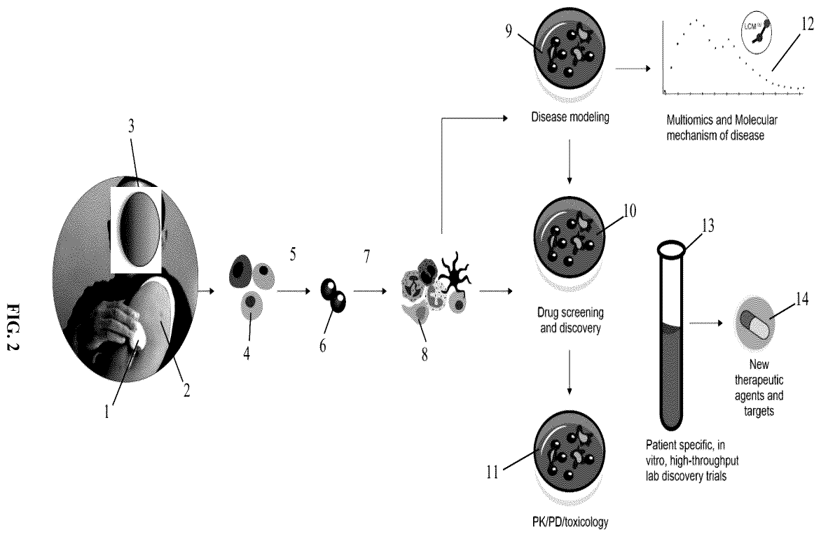

On the other hand, the FIG. 2 illustrates a comprehensive flow diagram of the Capillary Stem Cell Reprogramming (CSCR) system and method according to one embodiment of the present invention. The workflow depicted in FIG. 2 represents the complete process from initial capillary blood collection through downstream applications, encompassing multiple integrated steps that collectively enable patient-specific in vitro modeling and therapeutic discovery.

The process may be initiated with an upper-arm blood collection device (1) applied to the upper arm (2) of a person or user (3). The upper-arm blood collection device (1) may be selected from commercially available devices including, but not limited to, Tasso, YourBio Health TAP Micro, RedDropDx, and Impress devices. These devices may employ microfluidic channels, microneedles, or other minimally invasive mechanisms to access capillary blood vessels beneath the epidermis without requiring traditional venipuncture or phlebotomist intervention. The device (1) may be configured to collect a predetermined volume of capillary blood, typically ranging from approximately 100 μL to 1 mL, though the exact volume may vary depending on the specific application requirements.

Following collection, the capillary blood sample may be processed to isolate capillary peripheral blood mononuclear cells (cPBMCs) (4). This isolation step may utilize density gradient centrifugation, paramagnetic bead separation of cells, magnetic-activated cell sorting (MACS), or other separation techniques specifically optimized for capillary blood samples. The isolation protocol may be modified from traditional PBMC isolation methods to accommodate the smaller volume and potentially different cellular composition of capillary blood compared to venous blood. The isolation process may incorporate specialized reagents designed to maximize cPBMC yield and viability from the limited starting material. The system may employ automated isolation technologies to ensure consistent and reproducible results across different samples.

The isolated cPBMCs (4) may subsequently undergo a reprogramming step (5) to generate induced pluripotent stem cells (iPSCs) (6). This reprogramming process may utilize temporal mechanoporation or non-integrative methods such as Sendai virus vectors, episomal plasmids, or synthetic modified mRNA to introduce reprogramming factors including, but not limited to, OCT4, SOX2, KLF4, MYC, and LIN28. The reprogramming protocol may be specifically optimized for cPBMCs, with adjustments to factor concentrations, timing, and culture conditions that address the unique characteristics of cells derived from capillary rather than venous blood. The reprogramming step (5) may be performed under defined, xeno-free conditions to maintain clinical compatibility. The system may incorporate real-time monitoring of reprogramming efficiency through reporter systems or molecular markers to ensure quality control throughout the process.

The generated iPSCs (6) may retain the complete genetic and epigenetic profile of the person (3) from whom the capillary blood was collected, thereby preserving patient-specific characteristics that are essential for downstream personalized applications. The iPSCs (6) may be subjected to comprehensive quality control measures, including but not limited to, pluripotency marker assessment, karyotype analysis, and genomic sequencing to verify their integrity and suitability for subsequent applications.

The patient-specific iPSCs (6) may then undergo differentiation (7) into specific cell types of interest. This differentiation step may employ established protocols for generating diverse cell lineages including, but not limited to, neurons, cardiomyocytes, hepatocytes, or immune cells, depending on the intended application. The differentiation protocols may be optimized for iPSCs derived from cPBMCs, potentially incorporating small molecules, growth factors, or three-dimensional culture systems to enhance efficiency and functionality. The differentiation step (7) may be performed with precise temporal control to recapitulate developmental processes and produce mature, functional differentiated cells (8).

The reprogramming step (5) and differentiation step (7) may be collectively referred to as cell therapies within the context of the present invention, as they represent the critical cellular engineering components of the workflow. These steps may be performed under controlled conditions with defined media formulations and environmental parameters to ensure reproducibility and standardization across different patient samples.

The differentiated cells (8) generated through this process may proceed along one or both of two principal pathways. In the first pathway, the cells may be utilized directly for disease modeling (9), wherein patient-specific cellular phenotypes relevant to particular disease states may be recapitulated in vitro. This disease modeling step (9) may incorporate techniques such as CRISPR/Cas9-mediated genome editing to introduce or correct disease-associated mutations, thereby enabling the study of specific genetic variants in an isogenic background. The disease models may be developed in two-dimensional monolayer cultures, three-dimensional organoids, or microfluidic organ-on-chip systems to better approximate in vivo tissue architecture and functionality.

Alternatively or subsequently, the differentiated cells (8) may be directed to drug screening and discovery applications (10). This step may utilize the patient-specific differentiated cells in high-throughput or high-content screening platforms to evaluate compound libraries for efficacy, toxicity, or mechanism of action. The drug screening process may employ automated liquid handling, image-based phenotypic analysis, or reporter-based assays to quantify cellular responses to pharmaceutical agents. The screening platforms may be designed to accommodate multiple patient-derived cell lines simultaneously, enabling comparative analyses across different genetic backgrounds.

The disease modeling step (9) may likewise feed into the drug screening and discovery process (10), wherein disease-relevant phenotypes established in the modeling step may serve as the basis for therapeutic screening assays. This integration may allow for the identification of compounds that specifically target disease mechanisms rather than generalized cellular processes.

The drug screening and discovery step (10) may proceed to pharmacokinetic, pharmacodynamic, and toxicology analyses (11). This step may involve detailed characterization of compound absorption, distribution, metabolism, and excretion properties in patient-specific cellular systems. The toxicology component may assess acute and chronic cellular responses to compound exposure, potentially incorporating multi-organ systems to evaluate tissue-specific toxicities and organ interactions. Advanced analytical techniques such as high-resolution mass spectrometry or metabolic flux analysis may be employed to characterize drug metabolism in detail.

Independently, the disease modeling step (9) may proceed to multiomics and molecular mechanism investigations (12). This branch of the workflow may employ genomic, transcriptomic, proteomic, and metabolomic analyses to comprehensively characterize disease processes at the molecular level. Single-cell sequencing technologies may be applied to resolve cellular heterogeneity within patient-derived cultures. Computational approaches may integrate multimodal data to identify key regulatory networks and potential therapeutic targets underlying disease pathophysiology.

The collective outputs from these various analytical branches culminate in patient-specific, in vitro, high-throughput laboratory discovery trials (13). This comprehensive approach may enable the testing of hundreds or thousands of experimental conditions on patient-derived cells, effectively creating a “clinical trial in a dish” that preserves the unique molecular characteristics of the individual patient. The integration of artificial intelligence and machine learning algorithms may enhance data analysis and pattern recognition across complex datasets generated through these high-throughput approaches.

Ultimately, the system and method may facilitate the identification of new therapeutic agents and targets (14) tailored to individual patient profiles or specific disease subtypes. These discoveries may include novel small molecules, biologics, gene therapy approaches, or combination therapies identified through the patient-specific screening process. The therapeutic targets may encompass previously unrecognized disease mediators revealed through the detailed molecular characterization of patient-derived cellular systems.

The entire workflow depicted in FIG. 2 may be supported by sophisticated data management systems that maintain sample identity, process parameters, and analytical results throughout all steps. Quality control checkpoints may be implemented at each transition to ensure process integrity and data reliability. The system may incorporate automation and standardized protocols to enhance reproducibility while accommodating the inherent biological variability across patient samples.

Through this integrated approach, the CSCR system and method may provide unprecedented capabilities for personalized medicine, enabling targeted therapeutic development based on individual patient biology derived from minimally invasive capillary blood samples obtained through upper-arm collection devices. The workflow may significantly improve upon traditional drug discovery and development approaches by incorporating patient specificity from the earliest stages of the process rather than as a retrospective consideration during clinical trials.

In one embodiment, the upper-arm blood collection device may incorporate a temperature-controlled storage compartment that maintains the collected capillary blood sample at a predetermined temperature range of 4-8° C. for up to 48 hours. This feature may enable sample collection in remote or resource-limited settings where immediate processing may not be feasible. The compartment may utilize phase-change materials or miniaturized Peltier cooling elements to maintain temperature stability without requiring external power sources.

In another embodiment, the system may incorporate a portable, automated PBMC isolation module specifically designed for small-volume capillary blood samples. This module may utilize a modified density gradient centrifugation protocol optimized for volumes as low as 100 μL, thereby addressing the challenges associated with processing limited capillary blood samples. The module may employ specialized microfluidic channels with precise dimensional characteristics to enhance separation efficiency while minimizing cell loss. The module may also employ a paramagnetic bead cell separation system at point of care.

The reprogramming module may, in some embodiments, employ a sequential introduction of reprogramming factors rather than simultaneous delivery. This approach may involve the initial introduction of OCT4 and SOX 2, followed by KLF 4 after 48 hours, and finally c-MYC after an additional 48 hours. This temporal control may enhance reprogramming efficiency for cPBMCs by more closely recapitulating the natural hierarchy of transcription factor activation during early embryonic development.

In yet another embodiment, the system may incorporate a cryopreservation module enabling long-term storage of either the isolated cPBMCs or the generated iPSCs. This module may utilize controlled-rate freezing protocols with defined cryoprotectant formulations optimized for cells derived from capillary blood. The cryopreservation capability may facilitate biobanking of patient-specific samples for future applications or repeated experimental analyses.

The differentiation protocols may be modified in certain embodiments to include mechanical stimulation in addition to biochemical factors. For cardiac differentiation, the system may incorporate cyclical stretch or electrical field stimulation to enhance cardiomyocyte maturation and functionality. Similarly, for neural differentiation, the system may apply controlled mechanical tension to developing axons to promote proper synapse formation and network establishment.

In an alternative embodiment, the disease modeling step may incorporate co-culture systems wherein patient-derived cells are cultured alongside other cell types to recapitulate tissue-level interactions. For example, patient-specific neurons may be co-cultured with astrocytes and microglia to more accurately model neurodegenerative diseases with significant glial involvement. These co-culture systems may be established in microfluidic devices that enable precise control over cellular positioning and medium exchange.

The drug screening module may, in some implementations, incorporate label-free detection technologies such as impedance-based cellular analysis or digital holographic microscopy. These approaches may enable real-time, continuous monitoring of cellular responses to therapeutic candidates without requiring fluorescent or luminescent reporters that might alter cellular physiology. The label-free detection systems may be particularly advantageous for long-term studies examining compound effects over multiple days or weeks.

For toxicology applications, the system may incorporate fluidically-linked multi-organ platforms wherein different patient-specific cell types representing various organ systems are cultured in separate but interconnected chambers. This arrangement may enable the assessment of compound metabolism by hepatocytes and subsequent toxicity to cardiac or neural cells, thereby more accurately modeling systemic drug effects than isolated single-tissue systems.

The multiomics analysis module may, in certain embodiments, integrate spatial transcriptomics or proteomics technologies that preserve information regarding the distribution of molecular markers within three-dimensional organoid structures. This spatial resolution may provide insights into disease mechanisms that involve cell-cell interactions or regional specialization within tissues, aspects that would be lost in homogenized bulk analysis approaches.

In an enhanced embodiment, the system may incorporate real-time monitoring of cellular metabolism through integrated sensors for oxygen consumption, extracellular acidification, or specific metabolite production. These measurements may provide functional readouts complementary to endpoint molecular analyses, enabling more comprehensive characterization of disease phenotypes or drug responses.

The artificial intelligence component may be expanded in some implementations to include reinforcement learning algorithms that iteratively optimize differentiation protocols or compound screening strategies based on accumulated experimental outcomes. This adaptive capability may progressively enhance system performance as more patient samples are processed through the workflow.

For certain applications requiring extended culture periods, the system may incorporate automated medium exchange and monitoring systems that maintain optimal culture conditions with minimal manual intervention. These systems may employ sensors for key parameters such as pH, dissolved oxygen, and nutrient concentrations, with feedback-controlled medium replenishment to maintain homeostasis.

In another alternative embodiment, the system may utilize transient transfection of modified mRNA encoding reprogramming factors rather than viral vectors. This approach may offer enhanced safety profiles for potential therapeutic applications by eliminating any risk of genomic integration while maintaining efficient reprogramming. The mRNA delivery may be optimized through lipid nanoparticle formulations specifically designed for cPBMC transfection. Another delivery approach may employ circularized RNA that is introduce via mechanoporation.

The disease modeling applications may be extended in some embodiments to include aging-related phenotypes through telomere attrition protocols or progerin expression. These modifications may enable the study of age-dependent disease manifestations or drug responses, particularly relevant for chronic neurodegenerative or cardiovascular conditions that typically affect older populations.

In certain implementations, the system may incorporate microbiome components wherein patient-derived gut organoids are co-cultured with defined bacterial communities to study host-microbiome interactions in disease contexts. This approach may be particularly valuable for gastrointestinal disorders or conditions with significant microbiome involvement such as inflammatory bowel disease or certain neurological conditions.

The drug screening module may be adapted in some embodiments to evaluate cell-penetrating peptides or other delivery vehicles concurrently with therapeutic candidates. This capability may facilitate the identification of optimal drug-delivery combinations tailored to specific patient cellular characteristics, potentially enhancing therapeutic efficacy through improved intracellular compound delivery.

### INDUSTRIAL APPLICATION

The invention has significant industrial application in the fields of biotechnology, pharmaceuticals, and personalized medicine. By enabling the generation of patient-specific induced pluripotent stem cells (iPSCs) from minimally invasive capillary blood samples, the system streamlines drug discovery, disease modeling, and therapeutic development. Pharmaceutical companies can utilize the technology for high-throughput drug screening, toxicology analysis, in vitro clinical trials, and pharmacokinetic/pharmacodynamic studies, reducing reliance on animal models and improving predictive accuracy. Additionally, the invention supports the development of personalized therapeutics by providing patient-specific iPSCs for tailored drug testing and treatment optimization. Its integration with AI and multiomics further enhances its utility in advanced research and clinical applications, driving innovation in regenerative medicine and precision healthcare.

What is claimed is:

1. A system for generating patient-specific induced pluripotent stem cells (iPSCs) from capillary blood, comprising:
a) an upper-arm blood collection device configured to collect capillary blood from a patient, wherein the upper-arm blood collection device is selected from the group consisting of Tasso, Yourbio Health TAP Micro, RedDropDx, and Impress;
b) a processing module configured to isolate peripheral blood mononuclear cells (PBMCs) from the collected capillary blood;
c) a reprogramming module configured to reprogram the isolated PBMCs into iPSCs using a defined set of reprogramming factors; and
d) an in vitro modeling module configured to utilize the generated iPSCs for patient-specific disease modeling, drug discovery, or therapeutic applications.

2. The system of claim 1, wherein the upper-arm blood collection device is configured to collect capillary blood without the need for venipuncture or phlebotomist intervention.

3. The system of claim 1, wherein the processing module comprises a paramagnetic bead separation unit and a cell separation reagent to isolate PBMCs from the capillary blood.

4. The system of claim 1, wherein the reprogramming module is configured to introduce reprogramming factors selected from the group consisting of OCT4, SOX2, KLF4, MYC, and LIN28.

5. The system of claim 1, wherein the in vitro modeling module is configured to conduct patient-specific disease modeling, drug screening, or toxicology analysis using the generated iPSCs.

6. The system of claim 1, wherein the in vitro modeling module is further configured to integrate artificial intelligence (AI) for predictive analysis of drug efficacy, pharmacokinetics, or pharmacodynamics.

7. The system of claim 1, wherein the generated iPSCs retain the molecular characteristics of the patient from whom the capillary blood was collected.

8. The system of claim 1, wherein the in vitro modeling module is configured to perform multiomics analysis, including genomics, transcriptomics, proteomics, and metabolomics, on the generated iPSCs.

9. A method for generating patient-specific induced pluripotent stem cells (iPSCs) from capillary blood, comprising:
a) collecting capillary blood from a patient using an upper-arm blood collection device, wherein the upper-arm blood collection device is selected from the group consisting of Tasso, Yourbio Health TAP Micro, RedDropDx, and Impress;
b) isolating peripheral blood mononuclear cells (PBMCs) from the collected capillary blood;
c) reprogramming the isolated PBMCs into iPSCs using a defined set of reprogramming factors; and
d) utilizing the generated iPSCs for patient-specific in vitro modeling, drug discovery, or therapeutic applications.

10. The method of claim 9, wherein the capillary blood is collected without the need for venipuncture or phlebotomist intervention.

11. The method of claim 9, wherein the step of isolating PBMCs comprises centrifuging the capillary blood and separating PBMCs using a cell separation reagent.

12. The method of claim 9, wherein the reprogramming factors are selected from the group consisting of OCT4, SOX2, KLF4, MYC, and LIN28.

13. The method of claim 9, wherein the generated iPSCs are used for patient-specific disease modeling, drug screening, or toxicology analysis.

14. The method of claim 9, wherein the generated iPSCs are used for predictive analysis of drug efficacy, pharmacokinetics, or pharmacodynamics using artificial intelligence (AI).

15. The method of claim 9, wherein the generated iPSCs retain the molecular characteristics of the patient from whom the capillary blood was collected.

16. The method of claim 9, further comprising performing multiomics analysis, including genomics, transcriptomics, proteomics, and metabolomics, on the generated iPSCs.

17. The method of claim 9, wherein the in vitro modeling is used to predict safety and efficacy endpoints for clinical trials.

18. The method of claim 9, wherein the in vitro modeling is used for personalized therapeutics modeling.
